(19) United States
(12) Patent Application Publication
(10) Pub. No.: US 2018/0028724 A1
(43) Pub. Date: Feb. 1, 2018

(54) MATERIAL FOR THE MOLDING OF DEVICES TO BE IMPLANTED INTO THE HUMAN BODY OR OF ARTICULAR SPACERS

(71) Applicant: TECRES S.p.A., Sommacampagna (Verona) (IT)

(72) Inventors: Giovanni FACCIOLI, Monzambano (MN) (IT); Renzo SOFFIATTI, Nogara (VR) (IT)

(21) Appl. No.: 15/551,497

(22) PCT Filed: Feb. 16, 2016
(86) PCT No.: PCT/IB2016/050819

§ 371 Date: Aug. 16, 2017

(30) Foreign Application Priority Data
Feb. 16, 2015 (IT) VR2015A000022

(51) Int. Cl. A61L 27/54; A61L 27/12; B33Y 80/00; A61L 27/18; B33Y 10/00; B33Y 70/00; A61L 27/58; A61L 27/16

(52) U.S. Cl. CPC A61L 27/54; A61L 27/58; A61L 27/12; A61L 27/16; A61L 27/18; B33Y 10/00; B33Y 70/00; B33Y 80/00; A61L 2300/406; A61L 2300/104; A61L 2300/23; A61L 2300/102; A61L 2430/02; A61L 2430/24

(57) ABSTRACT

Material biocompatible and implantable in the human body, for the obtainment of a device that can be implanted in the human body or a spacer device for treating a bone or a joint seat, including an acrylic resin or a plastic material or polyethylene (PE) or low density polyethylene or high density polyethylene or ultra high molecular weight polyethylene (UHMWPE) or polypropylene or polyamide or polyetheretherketone (PEEK) or a mixture thereof, wherein the material can be moulded and includes an additive such as a pharmaceutical or medical substance and/or a radio-opacifying agent and/or a further additive; device that can be implanted in the human body or a spacer device for treating a bone or a joint location obtained with the aforementioned material and method for obtaining the material according to the present invention.

### TECHNICAL FIELD OF THE INVENTION

The present invention concerns a biocompatible and implantable material to be used to mould devices able to be implanted in the human body such as catheters, spacer devices, prostheses, etc.

### STATE OF THE ART

The technology of additive production or three-dimensional printing of objects is currently becoming increasingly popular.

In particular, three-dimensional or 3D printing, which represents a natural evolution of 2D printing, allows an object to be reproduced from a three-dimensional model through the superimposition of successive layers of material, until the desired shape is obtained. Such a three-dimensional model is obtained through suitable software.

3D printers are generally faster, more reliable and simpler to use with respect to other technologies for additive production and, moreover, offer the possibility of moulding and assembling parts made up of different materials with different physical and mechanical properties in a single construction process.

The operation of a three-dimensional printer is based on the use of a 3D file developed by suitable software.

The 3D model of the object of interest is prepared—through the aforementioned software—in a series of portions or layers in cross section thereof.

Each portion or layer is then printed one onto the other, in order to recreate the entire three-dimensional object.

There are different 3D printing technologies; their main differences concern the way in which the various layers are printed.

Some methods, in order to produce the various layers, use materials that melt or soften. Some examples of such technology are “Selective Laser Sintering” (SLS) or “Direct Metal Laser Sintering” (DMLS) or “Fused Deposition Modeling” (FDM).

Other methods provide for the deposition layer-by-layer of liquid materials, which are made to set with different technologies. An example of such a technology is known as “Digital Light Processing” (DLP).

Furthermore, in the case of lamination systems there are thin layers that are cut according to the desired shape and then joined together through known techniques.

A first 3D printing method consists of a printing system by extrusion of material. The printer creates the model one layer at a time, spreading a layer of powder (plaster or resins) and distributing a binder thereupon, for example printing like with an inkjet.

The process is repeated layer by layer until the desired shape of the object is obtained.

In this way, it is possible to make projections in the finished product.

FDM technology, used in conventional quick prototyping, provides for a nozzle adapted for depositing a molten polymer layer-by-layer on a support structure.

Furthermore, some 3D printers for additive synthesis use a thread of thermoplastic polymer as construction material.

In the field of orthopaedics, three-dimensional printing technology has led to bone or parts thereof being made. For example, it has been possible to implant a cranium printed in 3D to a patient.

The skullcap was made with a special resin through the use of a 3D printer. Other recently-developed prostheses were obtained through three-dimensional printing of a titanium-based material.

The materials usually used for 3D printing are: plastic materials, for example thermoplastic polymers (for example for SLS and FDM), metals, sand, glass (for example for SLS), photopolymers (for example for stereolithography), laminated sheets (often of the paper type) and relative glues, titanium alloys (for example for “Electron beam melting” or EBM), resins, clays, ceramic, etc.

In particular, for jet or thread 3D printing, the material used must be melted and extrudable through a nozzle suitable for the purpose.

Therefore, one of the problems to be solved in order to be able to three-dimensionally print some objects, particularly for medical or orthopaedic use, is precisely that of making materials having the appropriate characteristics for the final purpose for which the object must be made and at the same time characteristics suitable for the selected three-dimensional printing method.

Basically, surgical practice provides for implanting resins or plastic materials in the body for a short time (for example for catheters) or permanently (for example for polymethyl methacrylate or PMMA, constituent of bone cement, for ultra high molecular weight polyethylene or UHMWPE or in short PE, as friction surface in hip, knee, shoulder prostheses etc., or for polyetheretherketone or PEEK, as cranial prostheses, spinal cages, etc.).

Except for the PMMA of bone cement, all of the other plastic materials undergo mechanical processing in order to obtain a prosthesis or a device to be implanted in the human body.

This occurs both due to objective difficulty in moulding those specific plastic materials, and particularly for the extremely heterogeneous demand of prostheses or devices to be implanted in the human body of greatly different shape and size, requirements which only mechanical processing can quickly satisfy.

However, these plastic materials (for example PE and PEEK) do not and cannot contain pharmaceutical or medical substances, like for example one or more antibiotics, nor radio-opacifying agents, like for example barium, nor possible further medical additives used to treat the patient or for surgical treatment.

Such substances, however, would be very useful and appreciated inside such plastics in order to carry out a healing function (for example local antibiotic) and be easily detected radiographically, as done on the other hand by antibiotic-loaded bone cement provided with radio-opacifying agents.

Therefore, there is a need for a biocompatible and implantable material, added to with pharmaceutical or medical substances and/or radio-opacifying agents and/or other useful additives, to be used for moulding, also three-dimensional, of devices to be implanted in the human body or spacer devices.

The application WO2012/007535 discloses a part for endosseous implantation molded through injection moulding of a material comprising a thermoplastic binder (preferably PEEK) that incorporate fibers; TCP and zeolite can be incorporated into the binder, the latter substance being able to confer radiopacity at the implant.

The application EP0472237 discloses a material comprising UHMWPE and inorganic filler such as calcium phosphate or hydroxyapatite.

The application DE102007052519 discloses a medical implant, comprising a polymeric material (such as polyethylene or polypropylene) and an antimicrobial composition (including silicon dioxide and metal-containing nanoparticles).

The application WO2010/096053 discloses a medical implant incorporating a medical substance, such as silver or penicillin.

The application WO2013/184010 discloses a middle ear prosthesis including silver powder.

The U.S. Pat. No. 6,641,831 discloses a non-degradable medical product comprising at least two substances: substance A and substance B, wherein substance A is more lipophilic than substance B and has a given solubility in water, lower than that of substance B. At least one, among substance A and substance B, is a pharmaceutically active agent, i.e. an antimicrobial substance.

### SUMMARY OF THE INVENTION

The task of the present invention is to improve the state of the art.

In such a technical task, an aim of the present invention is to provide a biocompatible and implantable material, adapted for being used in moulding technology, possibly also three-dimensional printing, and which can be added to with pharmaceutical or medical substances and/or with further additives. An aim of the present invention is to provide a biocompatible and implantable material that is extrudable.

In accordance with an aspect of the present invention, a biocompatible and implantable material is provided according to the present application.

In accordance with another aspect of the present invention, a device to be implanted in the human body or a spacer device for the treatment of a bone or joint location made with a biocompatible and implantable material is provided, according to the present application.

An advantage of such a device to be implanted in the human body or a spacer device for the treatment of a bone or joint seat consists of being able to be added to with pharmaceutical or medical substances or with additives and be made through moulding, possibly three-dimensional moulding.

A further advantage of the device to be implanted in the human body or a spacer device for the treatment of a bone or joint location consists of being able to be personalised or made in series in a quick and simple manner, substantially without the need for further surface finishing processing.

In accordance with another aspect of the present invention a method for obtaining a biocompatible and implantable material is provided, adapted for being used in printing technology, possibly also three-dimensional printing, and which can be added to with pharmaceutical or medical substances and/or with a radio-opacifying agent and/or with further additives, according to the present application.

An advantage of such a method is that it is quick and simple, substantially without the need for further surface finishing processing steps.

The present application refers to preferred and advantageous embodiments of the invention.

### DETAILED DESCRIPTION OF THE INVENTION

The present invention refers to a material biocompatible and implantable in the human body, for the obtainment of a device that can be implanted in the human body or a spacer device for treating a bone or a joint seat.

Such a material can comprise one or more of the following materials: an acrylic resin or a plastic material or polyethylene (PE) or low density polyethylene or high density polyethylene or ultra high molecular weight polyethylene (UHMWPE) or polypropylene or polyamide or polyetheretherketone (PEEK), or a mixture thereof.

Such plastic materials, in a version of the invention, are insoluble.

In a further version of the invention, the plastic material is soluble and comprises, for example, polylactic or polyglycolic acid polymers or other suitable polymers.

Possible acrylic resins include an acrylic copolymer made up of MMA, styrene and ethyl-acrylate or polymethyl methacrylate or mixtures comprising acrylic polymers and/or copolymers.

One of the main characteristics of such a material is that it is mouldable, for example through injection moulding or through three-dimensional printing or through forming presses or through thermoplastic moulding technology.

In this way, especially when it concerns three-dimensional printing, it is possible to mould such a material according to a simple and quick procedure, capable of providing both medical devices or spacer devices produced in series, and personalised devices.

In the latter case, indeed, it is possible to obtain a three-dimensional model of the device to be obtained, by selecting the size and shape or configuration most suitable for the surgical or anatomical requirements of the patient, and produce the relative device adapted to the specific patient.

In the aforementioned cases, however, thanks to the mouldability of the material according to the present invention, it is possible to obtain finished devices, substantially without the need to carry out further surface finishing or processing steps thereof.

The material according to the present invention also comprises at least one additive such as a pharmaceutical or medical substance and/or a radio-opacifying agent and/or a further additive. The pharmaceutical or medical substance comprised in the material according to the present invention can consist of at least one antibiotic, for example gentamicin sulphate or another suitable antibiotic, or an antiseptic agent, of organic or inorganic nature, a bacteriostatic agent, like for example silver in its various forms, such as metallic powder or salts such as citrate, proteinate, colloidal, electrolytic, or other forms that can be used in the human body, etcetera.

For example, if the antiseptic agent or the bacterio static agent is metallic silver, this is insensitive to the melting temperature of the plastic material that would receive it according to the present invention.

Moreover, variously salified silver, for example colloidal silver powder, is insensitive to the melting temperature only of a few plastic materials of the material according to the present invention.

Possible bacteriostatic agents also comprise copper and/or gold, as well as the aforementioned silver, in their various forms, for example their salts or components. Such materials are, indeed, thermostable. Other thermostable inorganic substances having a medicating action can be advantageously included in the molten plastic material. A further example is boric acid that has an antiseptic action and is thermostable at over 300° C.

Such an option is particularly relevant when the device to be obtained with said material is a spacer device for treating an infection present in a bone or joint location.

The function of spacer devices, indeed, is to maintain the joint space left by an infected prosthesis, which for this reason is removed, and at the same time to treat the infection of the bone location, internally comprising for example a pharmaceutical or medical substance, like for example at least one antibiotic, to be eluted in the area to be treated.

The material according to the present invention, alternatively or in addition, could also comprise a radio-opacifying agent, like for example metallic powders, for example tungsten, tantalum, silver or salts such as barium sulphate, zirconium oxide, bismuth oxide, etcetera.

Such agents, as known, are visible to X-rays and thus make it possible to monitor the position of the device to be implanted in the human body or the spacer device, as well as the material that contains them according to the present invention.

The material according to the present invention, moreover, can comprise further medical additives, like for example soluble and/or reabsorbable ceramic material, in the form of powder or granules, such as tricalcium phosphate or calcium sulphate or hydroxyapatite, etcetera, or colouring substances of the biocompatible type and adapted to be introduced in the human body, etcetera.

Such additives, if they are not soluble or reabsorbable, can stay permanently inside the human body, or be removed if the biocompatible and implantable material in which they are contained is removed.

One of the problems to be solved, for the material according to the present invention, is that the substances contained therein can be degraded or undergo modifications due to the temperatures and/or pressures that the material according to the present invention encounters, when the material is heated or when it is moulded, for example through injection moulding or three-dimensional printing or another moulding technique.

In particular, the material according to the present invention can be in the form of a thread, for example having a diameter that can vary between 1 and 10 millimetres or between 1 and 5 millimetres, for example adapted for being used to feed a three-dimensional printer of the thread or inkjet type or in another moulding technique.

One of the methods for allowing the material according to the present invention to be reduced to a thread adapted for being extruded and/or injected and/or printed three-dimensionally is as follows.

The base material is provided, for example in the form of a pellet (e.g. press pellet) or of granules; the material is inserted in suitable machinery, for example an extruder, and an additive such as at least one from a pharmaceutical or medical substance and/or a radio-opacifying agent and/or a further additive is added.

Then the whole thing is heated until a certain temperature is reached, for example the melting temperature thereof, or to a temperature suitable for melting or softening the material in question (together with the possible mixture of additives that are added), to such a point as to be able to be extruded in threads or mouldable.

Such a melting temperature varies as the polymers comprised according to the present invention varies. Most of such polymers have melting values comprised between 60° and 300°. Such a melting temperature can, for example, be around 250° C.

In a version of the invention, there is then a step of extruding the heated or molten material through suitable machinery, for example the same extruder, in the form of one or more threads having the diameter indicated above.

Such at least one thread can be wound in a coil so that, being extruded, it cools down and becomes consolidated and thus is suitable to then be handled and stored.

Such at least one thread can be used to feed a nozzle of an injection mould or a 3D printer.

Such a thread can be moulded through a three-dimensional printer or through injection moulding or through forming presses or through a thermoplastic moulding technique, in order to obtain a device that can be implanted in the human body or a spacer device for treating a bone or a joint location.

Such a method can, additionally or as an alternative to those described above, provides for a step of crushing or granulation of the base material, possibly after cooling thereof.

In a version of the invention, such a step is carried out through a suitable granulating machine.

Thereafter, the base material, or the crushed or granulated material, is used in a thermoplastic press with which moulded products are obtained.

In this case, in a version, a thread is not obtained before moulding, whereas in a second version, it is the thread obtained as indicated earlier that is crushed or granulated, possibly after cooling thereof.

The crushed or granulated material is then moulded, for example through an injection moulding press.

As stated, the temperature at which the material is melted must be below the degradation or damaging temperature of the pharmaceutical or medical substance, or of the radio-opacifying agent or of the further additive, so that they maintain their original characteristics also, following melting of the material, in the thread or resulting material.

Therefore, in a version of the invention, the extrusion or the moulding (but not the 3D printing) is carried out at lower temperatures than that at which the aforementioned substances degrade, by suitably selecting plastic materials with particularly low melting or softening temperatures, for example below the melting or degradation temperature of the pharmaceutical or medical substance, in order to obtain the end product comprising at least one from a pharmaceutical or medical substance, or a radio-opacifying agent or a further additive, according to the specific requirements.

For example, an antibiotic that has a melting temperature of 180° C., so as not to degrade during moulding, must not exceed such a temperature.

Through the aforementioned material it is possible to obtain devices that can be implanted in the human body like for example catheters, for which it may be useful to have a medicated version, a non-medicated version, an X-ray visible version, a coloured version, etcetera.

Alternatively, through the aforementioned material it is possible to obtain devices that can be implanted in the human body like for example medical “threads”, for which it may be useful to have a medicated version, for example comprising an antibiotic and/or a radio-opaque substance.

In a version of the invention, the material is adapted for making devices that can be implanted in the human body or spacer devices initially without pharmaceutical or medical substances but capable of absorbing such substances at a later time, after they have been moulded.

In this way, if the material in question requires a melting temperature to obtain it in the form of thread or a moulding temperature above the degradation temperature of the pharmaceutical or medical substance, it will equally be possible to obtain the device in question, and insert the substances of interest at a later time, without the risk of damaging them. In this case, the device in question is porous. The porosity of the device makes it capable of absorbing, for example by capillary action, such substances after it has been formed.

In a further version, such devices are made with a material already added to with at least one pharmaceutical or medical substance but, being porous, once formed they can be capable of absorbing another substance, the same or different with respect to the one already contained in them.

In this way, it has been seen how the material according to the present invention, being able to be moulded and possibly being able to be made in the form of a thread, allows devices able to be implanted in the human body or spacer devices to be obtained in a quick, simple and possibly personalisable manner both in terms of the shape and the size, and also the pharmaceutical or medical substances or the further additives or agents contained therein, according to the surgical or anatomical requirements of the patient.

The present invention also refers to a device that can be implanted in the human body or to a spacer device for treating a bone or a joint location, comprising a material biocompatible and implantable in the human body according to the present invention.

Such devices, in fact, comprise an additive such as a pharmaceutical or medical substance and/or a radio-opacifying agent and/or a further additive, as described earlier for the material according to the present invention.

Such devices are made by moulding, for example through a three-dimensional printer or through injection moulding or through forming presses or through a thermoplastic moulding technique.

Another embodiment that can be obtained with the material according to the present invention is a cranial prosthesis. In this way, the prosthesis could be made directly from the CAT data, formed according to the configuration and the dimensions necessary for the anatomical and implanting requirements, and then implanted in the bone location of interest.

Such a cranial prosthesis could contain a pharmaceutical or medical substance and/or a radio-opacifying agent and/or a further additive.

The invention thus conceived can undergo numerous modifications and variants all covered by the inventive concept.

The characteristics presented for one version or embodiment can be combined with the characteristics of another version or embodiment, without departing from the scope of protection of the present invention.

Moreover, all of the details can be replaced by other technically equivalent elements. In practice, the materials used, as well as the contingent shapes and sizes, can be whatever according to requirements without for this reason departing from the scope of protection of the following claims.

1. Material biocompatible and implantable in the human body, for the obtainment of a device that can be implanted in the human body or a spacer device for treating a bone or a joint location, comprising a plastic material such as an acrylic resin or a plastic material or polyethylene (PE) or low density polyethylene or high density polyethylene or ultra high molecular weight polyethylene (UHMWPE) or polypropylene or polyamide or polyetheretherketone or a mixture of the above, characterized in that it comprises an additive such as a pharmaceutical or medical substance and/or a radio-opacifying agent and/or a further additive and in that said biocompatible material is moldable.

2. Material according to claim 1, wherein said biocompatible material is extruded or extrudable into a thread.

3. Material according to claim 2, wherein said thread has a diameter comprised between 1 and 10 mm or between 1 and 5 mm.

4. Material according to any one of the preceding claims, wherein said biocompatible material is a material moldable by means of a tridimensional printer or by means of injection molding or by means of molding presses or using a thermoplastic molding technique.

5. Material according to claim 1, wherein said pharmaceutical or medical substance comprises at least one antibiotic, one organic antibiotic, gentamicin sulphate or another suitable antibiotic, or an antiseptic agent of organic or inorganic nature, a bacteriostatic agent such as silver in its forms, such as metallic powder or salts such as citrate, proteinate, colloidal, electrolytic, or other forms that can be used in the human body, or copper or gold in their forms, such as metals, alloys or salts, boric acid, etcetera.

6. Material according to claim 1, wherein said radio-opacifying agent comprises tungsten, tantalum or silver metallic powders or their salts or barium sulphate, zirconium oxide, bismuth oxide, etcetera.

7. Material according to claim 1, wherein said further additive comprises a soluble and/or resorbable ceramic material, in form of powder or granules, such as tricalcium phosphate or calcium sulphate or hydroxyapatite, etcetera, or coloring substances of the biocompatible type and adapted to be introduced in the human body, etcetera.

8. Material according to claim 1, wherein said acrylic resin comprises an acrylic copolymer comprising MMA, styrene and ethyl acrylate or polymethylmethacrylate or mixtures comprising acrylic polymers and/or copolymers.

9. Device implantable in the human body or spacer device for treating a bone or a joint location, comprising a biocompatible material that can be implanted in the human body, wherein said biocompatible material comprises a plastic material such as an acrylic resin or polyethylene (PE) or low density polyethylene or high density polyethylene or ultra high molecular weight polyethylene (UHMWPE) or polypropylene or polyamide or polyetheretherketone (PEEK) or a mixture of the above, characterized in that said biocompatible material comprises an additive such as a pharmaceutical or medical substance and/or a radio-opacifying agent and/or a further additive.

10. Device according to claim 9, wherein said device is obtained by molding said biocompatible material by means of a tridimensional printer or by means of injection molding or by means, of molding presses or using a thermoplastic molding technique.

11. Device according to claim 9 or 10, wherein said pharmaceutical or medical substance comprises at least one antibiotic, one organic antibiotic, gentamicin sulphate or another suitable antibiotic, or an antiseptic agent of organic or inorganic nature, a bacteriostatic agent such as silver in its forms, such as metallic powder or salts such as citrate, proteinate, colloidal, electrolytic, or other forms that can be used in the human body, or copper or gold in their forms and alloys, or as salts, etcetera.

12. Device according to any one of the claims 9-11, wherein said radio-opacifying agent comprises tungsten, tantalum or silver metallic powders or salts such as barium sulphate, zirconium oxide, bismuth oxide, etcetera, and/or wherein said further additive comprises a soluble and/or reabsorbable ceramic material, in form of powder or granules, such as tricalcium phosphate or calcium sulphate or hydroxyapatite, etcetera, or coloring substances of the biocompatible type and adapted to be introduced in the human body, etcetera.

13. Device according to any one of the claims 9-12, wherein said acrylic resin comprises a copolymer composed of MMA, styrene and ethyl acrylate or polymethylmethacrylate or mixtures comprising acrylic polymers and/or copolymers.

14. Method for the obtainment of a biocompatible material that can be implanted in the human body according to any one of the claims 1-8, comprising the following steps:
providing a plastic material comprising an acrylic resin or a plastic material or polyethylene (PE) or low density polyethylene or high density polyethylene or ultra high molecular weight polyethylene (UHMWPE) or polypropylene or polyamide or polyetheretherketone (PEEK) or a mixture of the above, wherein said material can be molded,
providing an additive such as a pharmaceutical or medical substance and/or a radio-opacifying agent and/or a further additive,
mixing or adding said plastic material and said additive obtaining a mixed or added base material.

15. Method according to claim 14, comprising the following steps:
heating said mixed or added base material to a predetermined temperature and extruding of the melted material in shape of one or more threads, or
heating said mixed or added base material to a predetermined temperature, cooling it, crushing or granulating said material in order to obtain a crushed or granulated material, or
heating said mixed or added base material to a predetermined temperature, extruding the melted material in shape of one or more threads and crushing or granulating said material in shape of one or more threads in order to obtain a crushed or granulated material.

16. Method according to claim 14 or 15, comprising a step of molding said thread or said crushed or granulated material by means of a tridimensional printer or by means of injection molding or by means of molding press or using a thermoplastic molding technique, in order to obtain a device that can be implanted in the human body or a spacer device in order for treating a bone or a joint location.

17. Method according to claim 14 or 15, wherein said mixing or adding step and said heating step of said material occur simultaneously in a dedicated machinery, such as an extruder.

18. Thread for tridimensional printing comprising a material according to any one of the claims 1 to 8.

19. Method for the obtainment of a device implantable in the human body or spacer device for treating a bone or a joint location according to any one of claims 9 to 13, comprising the steps of:
obtaining a biocompatible material according to any one of claims 1 to 8, and
molding said biocompatible material.
